(19) United States
(12) Patent Application Publication
(10) Pub. No.: US 2026/0090889 A1
(43) Pub. Date: Apr. 2, 2026

(54) TIBIAL AUGMENT BLOCK

(71) Applicant: The Third Hospital of Changsha, Changsha City (CN)

(72) Inventors: Ting DENG, Changsha City (CN); Qing LEI, Changsha City (CN); Zhuo ZHANG, Changsha City (CN); Song CHEN, Changsha City (CN)

(21) Appl. No.: 19/044,886

(22) Filed: Feb. 4, 2025

(30) Foreign Application Priority Data
Sep. 27, 2024 (CN) 202411364890.4

(51) Int. Cl. A61F 2/28; A61F 2/30

(52) U.S. Cl. CPC A61F 2/28; A61F 2002/2892; A61F 2002/30736

(57) ABSTRACT

Provided is a tibial augment block, including an insert. The insert is arc-shaped, a convex side of the insert is provided with a connecting plate for being connected to an outer wall of tibia, a connecting hole facing the tibia is formed in the connecting plate, a certain included angle is formed between an axis of the connecting hole and each of upper and lower end surfaces of a tibial osteotomy area, and a connector for connecting the tibia is arranged in the connecting hole. The connecting hole is provided to make the connector form a certain angle with each of the upper and lower end surfaces of the osteotomy area.

### CROSS-REFERENCE TO RELATED APPLICATION

This patent application claims the benefit and priority of Chinese Patent Application No. 2024113648904, filed with the China National Intellectual Property Administration on Sep. 27, 2024, the disclosure of which is incorporated by reference herein in its entirety as part of the present application.

### TECHNICAL FIELD

The present disclosure relates to the technical field of medical devices, and in particular to a tibial augment block.

### BACKGROUND

With the popularization of knee arthroplasty in China and the improvement of the sensitivity of patients to pain, the number of knee arthroplasty is increasing year by year. At present, high tibial osteotomy (HTO) is the first choice for “knee preservation”. Osteotomy is often needed during surgery, and a wedge-shaped defect occurs on one side of the tibia. After osteotomy, an insert needs to be implanted in the osteotomy area. The insert can change the weight-bearing alignment of the affected limb, correct the uneven stress distribution on the knee joint to redistribute the stress on the knee joint surface, correct the poor alignment, and slow down the progress of joint lesions. Therefore, the stability of the connection between the insert and the tibia will directly affect the recovery speed after surgery.

Therefore, there is an urgent need of a tibial augment block capable of being stably connected to the tibia.

### SUMMARY

In order to solve the problem above, the present disclosure provides a tibial augment block. A connector may have a certain included angle with each of upper and end surfaces of a tibial osteotomy area through connecting holes, and the connection strength between the insert and the tibia is improved.

To achieve the objective above, the present disclosure employs the following technical solution:

A tibial augment block includes an insert. The insert is arc-shaped, a convex side of the insert is provided with a connecting plate for being connected to an outer wall of tibia, a connecting hole facing the tibia is formed in the connecting plate, a certain included angle is formed between an axis of the connecting hole and each of upper and lower end surfaces of a tibial osteotomy area, and a connector for connecting the tibia is arranged in the connecting hole.

Preferably, the insert includes a base surface in contact with a lower cut surface of the tibia, and a support surface in contact with an upper cut surface of the tibia. The base surface is horizontally arranged, and the support surface is inclined towards the base surface in a direction away from the connecting plate.

Preferably, an outer arc surface of the insert is in fit with the outer wall of the tibia.

Preferably, an extending direction of the connecting plate is consistent with an inclined direction of the outer arc surface.

Preferably, each of the base surface and the support surface is provided with a pore structure.

Preferably, both ends of the connecting plate extend to upper and lower sides of the insert, respectively. Two connecting holes are provided, and the two connecting holes are formed in both ends of the connecting plate, respectively.

Preferably, a thickness of the insert gradually decreases from middle to both ends.

Preferably, the middle of the insert has a thickness 6.0-20.0 mm, and both ends of the insert have a thickness 2.0-3.0 mm.

Preferably, a side surface, close to the tibia, of the connecting plate is arranged as an arc, and a radian of the connecting plate is in fit with that of the outer wall of the tibia.

Preferably, the axis of the connecting hole extends towards a direction away from an axis of the tibia.

Compared with the prior art, the present disclosure has the following technical effects:

In the tibial augment block provided by the present disclosure, a certain included angle is formed between the axis of the connecting hole and each of the upper and lower end surfaces of the tibial osteotomy area, such that a certain included angle is also formed between the connector and each of the upper and lower end surfaces of the osteotomy area. The connector is used for connecting the tibia and the insert. When the insert tends to move away from the tibia, a thrust perpendicular to an axial direction of the connector is generated, such that a frictional force between the connector and the tibia is greatly increased, and the connection strength between the connector and the tibia is improved, and then the insert is less likely to be separated from the osteotomy area, and the technical effect of improving the connection stability between the tibial augment block and the tibia is achieved.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a schematic diagram of an embodiment of a tibial augment block;

FIG. 2 is a schematic diagram of FIG. 1 from another perspective;

FIG. 3 is a schematic diagram of FIG. 2 from another perspective;

FIG. 4 is a schematic diagram of the fitting of a tibial augment block and tibia;

FIG. 5 is a schematic diagram of the fitting of a tibial augment block and tibia according to an embodiment.

In the drawings: 1—insert; 2—connecting plate; 3—connecting hole; 4—osteotomy area; 5—base surface; 6—support surface; 7—outer arc surface.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide a tibial augment block. Through a connector which forms a certain included angle with each of upper and lower end surfaces of a tibial osteotomy area, the connection strength between an insert and the tibia is improved.

In order to make the objectives, features and advantages of the present disclosure more clearly, the present disclosure is further described in detail below with reference to the accompanying drawings and specific embodiments.

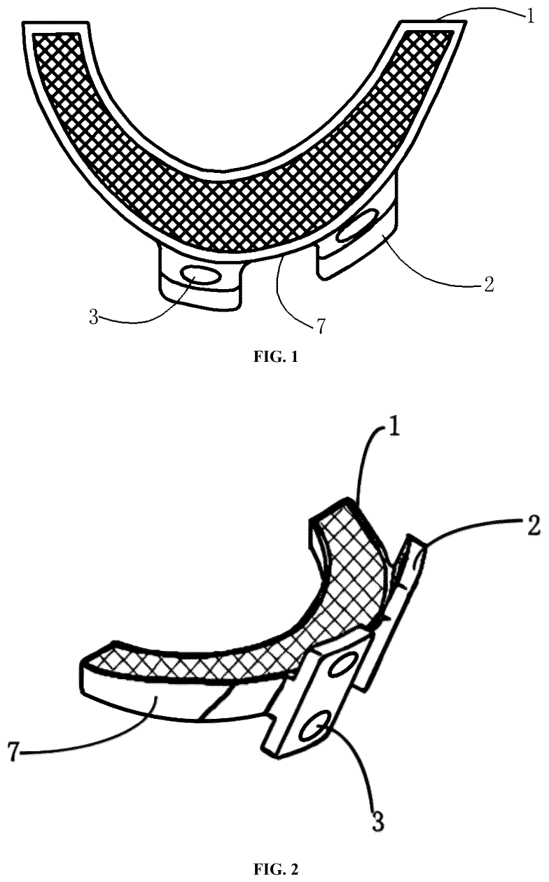

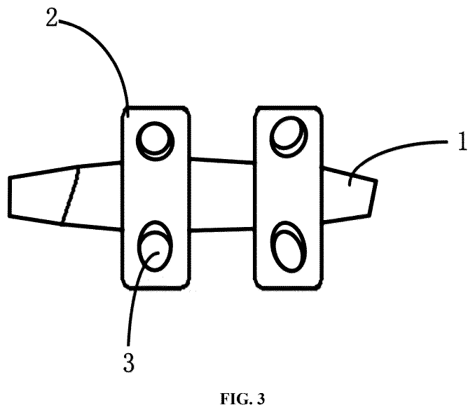

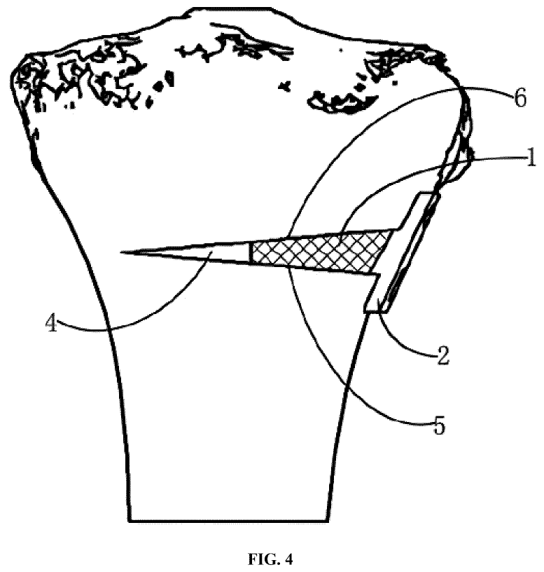

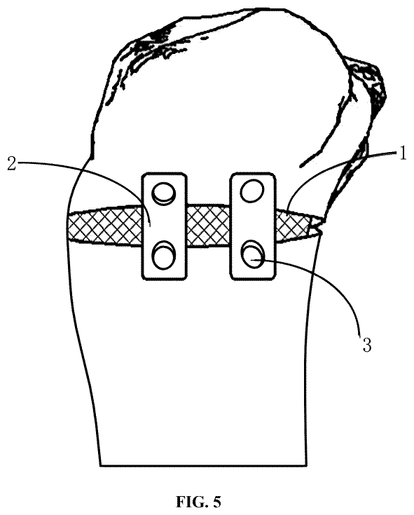

Referring to FIG. 1 to FIG. 5, a tibial augment block provided in the embodiment of the present disclosure includes an insert 1. The insert 1 is arc-shaped, a convex side of the insert 1 is provided with a connecting plate 2 for being connected to an outer wall of tibia, a connecting hole 3 facing the tibia is formed in the connecting plate 2, and a connector for connecting the tibia is arranged in the connecting hole 3. The connector is used for connecting the tibia and the insert. A certain included angle is formed between an axis of the connecting hole 3 and each of upper and lower end surfaces of a tibial osteotomy area 4, such that a certain included angle is also formed between the connector and each of the upper and lower end surfaces of the osteotomy area 4. No matter along which end surface the insert 1 tends to move away from the tibia, the insert 1 can generate a thrust perpendicular to an axial direction of the connector through the connecting plate 2, such that a frictional force between the connector and the tibia is greatly increased, the connection strength between the connector and the tibia is improved, and the insert 1 is less likely to be separated from the osteotomy area at an initial stage of the surgery, and the technical effect of improving the connection stability between the tibial augment block and the tibia is achieved. Meanwhile, there is a large space in the middle of the arc-shaped insert 1, such that the augment block does not need to be removed even if the artificial knee arthroplasty is needed later, which provides sufficient convenience for the follow-up treatment of a patient.

It may be understood that the connector may be a medical steel nail, a medical screw, and other known connectors. Preferably, the connector is a threaded self-tapping screw meeting the medical requirements, a diameter of which is preferably 3-4 mm.

As a preferred embodiment, the insert 1 includes a base surface 5, and a support surface 6. The base surface 5 is abutted against a lower cut surface of the osteotomy area 4, and the support surface 6 is abutted against an upper cut surface of the osteotomy area 4. The base surface 5 is horizontally arranged, and the support surface 6 is inclined towards the base surface 5 in a direction away from the connecting plate 2, such that the insert 1 fits the wedge-shaped osteotomy area 4 more closely. On the one hand, the discomfort of the patient is reduced, on the other hand, the connection stability between the insert 1 and the tibia is further improved.

More preferably, the connecting plate 2 has a thickness of 2-3 mm.

As a preferred embodiment, an outer arc surface 7 of the insert 1 is in fit with the outer wall of the tibia, which is located between the base surface 5 and the support surface 6, and connected to an outer edge of the base surface 5 and an outer edge of the support surface 6, respectively. The outer edge of the base surface 5 is closer to the axis of the tibia than the outer edge of the support surface 6, such that the outer arc surface 7 has an inclination angle inclined away from the axial direction of the tibia to fit the shape of the tibia.

More preferably, an extending direction of the connecting plate 2 is consistent with an inclined direction of the outer arc surface 7, such that the connecting plate 2 can be in fit with the shape with a big upper part and a small lower part, and then is closely attached outside the tibia.

As a preferred embodiment, each of the base surface 5 and the support surface 6 is provided with a pore structure, and the base surface 5 and the support surface 6 are in contact with the upper and lower end surfaces of the tibial osteotomy area 4, respectively. The pore structure is provided to make regenerated tibia grow into the pore structure, such that the tibia can be integrated with the insert 1, which is more conducive to the later bone integration function and ensures the connection strength between the insert 1 and the tibia in the later recovery process. It may be understood that the pore structure may be multiple openings and/or grooves formed in the base surface 5 and the support surface 6, which can be arranged uniformly or randomly according to actual needs.

Further, in order to further improve the connecting strength between the insert 1 and the tibia, two connecting plates 2 are provided.

As a preferred embodiment, as shown in FIG. 1 to FIG. 5, both ends of the connecting plate 2 extend to upper and lower sides of the insert 1, respectively. Two connecting holes 3 are provided, and the two connecting holes 3 are formed in both ends of the connecting plate 2, respectively. Preferably, an extending direction of the connector is perpendicular to a bone surface of the tibia. Correspondingly, an axial direction of the connecting hole 3 is also perpendicular to the bone surface at a corresponding position, such that the connection between the insert 1 and the tibia is more stable.

As a preferred embodiment, in order to improve the fitting degree between the insert 1 and the end surface of the tibia, a thickness of the insert 1 is arranged in a way of decreasing from middle to both ends. More specifically, the middle of the insert 1 has a thickness 6.0-20.0 mm, and both ends of the insert 1 have a thickness 2.0-3.0 mm.

As a preferred embodiment, as the outer wall of the tibia has a shape with a big upper part and a small lower part, the shape of the tibia is an arc with different curvatures. In order to make the connecting plate 2 closely attached to the tibia, a side surface, close to the tibia, of the connecting plate 2 is arranged as an arc, such that a radian of a contact surface between the connecting plate 2 and the tibia is in fit with that of the outer wall of the tibia, and the connection between the whole tibial augment block and the tibia is more stable.

As a preferred embodiment, the axial direction of the connecting hole 3 is parallel to a coronal plane of the insert 1, and the axial direction of the connecting hole 3 forms an inclination angle of 30°-40° with a horizontal plane of the insert 1. The coronal plane and the horizontal plane are anatomical terms, corresponding to the insert 1, the coronal plane refers to a cut surface that divides an anatomical position of the insert 1 into front and rear parts, and the horizontal plane refers to a cut surface that divides the anatomical position of the insert 1 into upper and lower parts.

As another embodiment, the axial direction of the connecting hole 3 extends towards a direction away from the axis of the tibia, thus preventing the size of the reserved space in the middle of the insert 1 from being affected by the arrangement of the connector.

More preferably, the connecting hole 3 is a counterbore hole, thereby avoiding a problem that a protruding end of the connector has adverse effects on the patient.

As a preferred embodiment, the tibial augment block is made of a tantalum metal material. More preferably, the tibial augment block is integrally formed by 3D printing technology.

Adaptive changes made according to actual needs are within the scope of protection of the present disclosure.

It should be noted that it is apparent to those skilled in the art that the present disclosure is not limited to the details of the above exemplary embodiments, and can be implemented in other specific forms without departing from the spirit or basic characteristics of the present disclosure. Therefore, the embodiments should be considered as exemplary and non-limiting in all aspects, and the scope of the present disclosure is defined by the appended claims rather than the above description, so it is intended to embrace all changes that fall within the meaning and range of equivalents of the claims. Any reference numerals in the claims should not be regarded as limiting the claims involved.

What is claimed is:

1. A tibial augment block, comprising an insert (1), wherein the insert (1) is arc-shaped, a convex side of the insert (1) is provided with a connecting plate (2) for being connected to an outer wall of tibia, a connecting hole (3) facing the tibia is formed in the connecting plate (2), a certain included angle is formed between an axis of the connecting hole (3) and each of upper and lower end surfaces of a tibial osteotomy area (4), and a connector for connecting the tibia is arranged in the connecting hole (3).

2. The tibial augment block according to claim 1, wherein the insert (1) comprises a base surface (5) in contact with a lower cut surface of the tibia, and a support surface (6) in contact with an upper cut surface of the tibia; the base surface (5) is horizontally arranged, and the support surface (6) is inclined towards the base surface (5) in a direction away from the connecting plate (2).

3. The tibial augment block according to claim 2, wherein an outer arc surface of the insert (1) is in fit with the outer wall of the tibia.

4. The tibial augment block according to claim 3, wherein an extending direction of the connecting plate (2) is consistent with an inclined direction of the outer arc surface (7).

5. The tibial augment block according to claim 2, wherein each of the base surface (5) and the support surface (6) is provided with a pore structure.

6. The tibial augment block according to claim 1, wherein both ends of the connecting plate (2) extend to upper and lower sides of the insert (1), respectively, two connecting holes (3) are provided, and the two connecting holes (3) are formed in both ends of the connecting plate (2), respectively.

7. The tibial augment block according to claim 2, wherein a thickness of the insert (1) gradually decreases from the middle to both ends.

8. The tibial augment block according to claim 7, wherein the middle of the insert (1) has a thickness 6.0-20.0 mm, and both ends of the insert (1) have a thickness 2.0-3.0 mm.

9. The tibial augment block according to claim 1, wherein a side surface, close to the tibia, of the connecting plate (2) is arranged as an arc, and a radian of the connecting plate (2) is in fit with that of the outer wall of the tibia.

10. The tibial augment block according to claim 1, wherein the axis of the connecting hole (3) extends towards a direction away from an axis of the tibia.
